(19) United States
(12) Patent Application Publication
(10) Pub. No.: US 2016/0101141 A1
(43) Pub. Date: Apr. 14, 2016

(54) Topical Treatment of Sports Related Injuries

(71) Applicant: Topical Biomedics, Inc., Rhinebeck, NY (US)

(72) Inventor: Lou Paradise, Rhinebeck, NY (US)

(21) Appl. No.: 14/514,348

(22) Filed: Oct. 14, 2014

(51) Int. Cl. A61K 36/81; A61L 15/44; A61K 36/28; A61K 9/00; A61K 36/77; A61K 36/22

(52) U.S. Cl. CPC A61K 36/81; A61K 36/77; A61K 36/22; A61K 36/28; A61K 9/0014; A61L 15/44; A61L 2300/402; A61L 2300/45; A61F 2250/0067

(57) ABSTRACT

The present invention relates to a homeopathic composition that may be used as a pain management treatment of musculoskeletal injuries. More particularly, the present disclosure and claims provide a treatment for sports related injuries (SRIs). In particular, the present invention provides a topical application, such as a cream or gel, which may be applied on or into the skin, wherein the topical application comprises one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution.

### FIELD OF INVENTION

The present invention relates to a homeopathic composition that may be used as a pain management treatment of musculoskeletal injuries. More particularly, the present disclosure and claims provide a treatment for sports related injuries (SRIs).

### BACKGROUND

Approximately seven million American seek care annually for sports related injuries (SRIs), and that number continues to rise. Most commonly, SRIs are musculoskeletal in nature. There are two primary types of SRIs: acute, wherein an injury may occur suddenly during the sport-related activity; and chronic, wherein the injury may develop after extensive sport-related activity. Acute SRIs, such as sprained ankles or fractured hands, may be characterized by sudden severe pain, swelling, tenderness, weakness, immobility, and/or visible dislocation, and combinations thereof. Chronic SRIs, such as tennis elbow, may be characterized by pain during activity, dull aching when resting, and/or swelling, and combinations thereof.

Currently, non-topical treatments may include oral pain relievers, cortisone injections, surgery, ultrasound, electrostimulation, physiotherapy, and general home care (rest, ice, compression, elevation, and/or immobilization). However, the primary function of many non-topical treatments is to decrease inflammation or to allow for more effective healing. Oral pain relievers may target pain symptoms but may cause adverse side effects and/or drug interactions.

Topical pain relief may have fewer adverse systemic effects than more invasive treatments and may allow for direct treatment of acute and chronic pain. Many available topical treatments for SRIs may include general use topical agents, such as lidocaine or capsaicin. More targeted topical treatments, such as diclofenac epolamine, may require a prescription.

Accordingly, what is needed therefore is a more effective and easily accessible topical treatment for SRIs.

### SUMMARY

Accordingly, the present invention provides a homeopathic composition that may be used as a pain management treatment of musculoskeletal injuries. More particularly, the present disclosure and claims provide a treatment for sports related injuries (SRIs). In particular, the present invention provides a topical application, such as a cream or gel, which may be applied on or into the skin, wherein the topical application comprises one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution.

The details of one or more embodiments of the invention are set forth in the accompanying drawings and the description below. The accompanying drawings that are incorporated in and constitute a part of this specification, illustrate several embodiments of the invention and, together with the description, serve to explain the principles of the invention: Other features, objects, and advantages of the invention will be apparent from the description, drawings and the claims herein.

### BRIEF DESCRIPTION OF THE DRAWINGS

The accompanying drawings, that are incorporated in and constitute a part of this specification, illustrate several embodiments of the invention and, together with the description, serve to explain the principles of the invention:

FIG. 1A illustrates an anterior view of a human body, wherein common sport related injury sites are highlighted.

FIG. 1B illustrates a posterior view of a human body, wherein common sport related injury sites are highlighted.

FIG. 2 illustrates exemplary musculature of the posterior of the human torso, wherein the trapezius muscle is highlighted.

FIG. 3 illustrates an exemplary nervous system of the human body, wherein the radial nerve is highlighted.

FIG. 4A illustrates an exemplary SRI pad with a homeopathic composition.

FIG. 4B illustrates an exemplary roll of SRI wrap with a homeopathic composition

FIG. 5 illustrates an exemplary treatment bandage.

### DETAILED DESCRIPTION

The present invention relates generally to a homeopathic composition that may be used as a pain management treatment of musculoskeletal injuries. More particularly, the present disclosure and claims provide a treatment for sports related injuries (SRIs). In particular, the present invention provides a topical application, such as a cream or gel, which may be applied on or into the skin, wherein the topical application comprises one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution.

As used herein, an SRI refers to any injury typically associated with sport participation, such as described in the background, though the actual cause of the injury may or may not be due to sport participation. For example, tennis elbow may be typically associated with playing tennis, though the injury may also occur in a work place from utilizing a computer. In either event, the injury may be referred to as an SRI, according to the present disclosure.

More specifically, the components of the composition may comprise a vasodilator, such as, for example, Arnica Montana, primrose oil, rosemary, ginger, Hamamelis, horse chestnuts, Yohimbe, niacin L-ornithate, and the like; a stimulator of lymphatic activity, such as, for example, Echinacea Augustofolia, St. Johns Wort, Belladonna, Devils Claw, Yellow Dock, Burdock, North American Ginseng, Wild Indigo, Pleurisy Root, and Pokeweed; and mobilizers of white blood cell activity, such as, for example, Lachesis Ninta venom, Crotalus horridus venom, Naja venom, Red Chinese Ginseng, Siberian Ginseng and Korean Ginseng, Goldenseal, Chamomile, and Barberry. Each of the components may be used in the amount of about 0.5 to 5%, preferably, 0.5 to 2.0% by weight.

In some embodiments, the homeopathic composition may comprise snake venom and venom extracts, which have anti-inflammatory capabilities. The snake venom may reduce swelling on or around sites of SRI.

In some aspects, the homeopathic composition may comprise prostaglandins, which may have been found to improve nerve conduction velocity. In some embodiments, prostaglandins may be formed to be advantageous to induce prostaglandin production at the site affected by the SRI rather than infusing the entire body. Accordingly, the inclusion of 0.5 to 5% by weight of the composition such as primrose oil, flax oil, and crocetin, may be beneficial.

In some embodiments, the homeopathic composition may comprise Caprylyl Glycol, which may function as a humectant and/or stabilizer. Caprylyl glycol may be plant derive or synthetic. Caprylyl glycol may increase antimicrobial activity of other preservatives, and may be used in conjunction with phenoxyethanol and chloroxylenol. The preservative blend may work on the skin's surface to give it a soft and smooth texture.

In some aspects, the homeopathic composition may comprise Caprylic/Capric Triglyceride, which may be produced by chemical reactions between various fatty acids and glycerol (synthetic glycerine). This largely synthetic mixed triester may be derived from coconut oil and glycerin and may comprise an emollient, dispersing agent, and solvent. In some aspects, Caprylic/Capric Triglyceride may comprise spreading properties that may facilitate gliding of the homeopathic composition on the skin.

In some embodiments, the homeopathic composition may comprise Caprylhydroxamic Acid, which may take place of traditional preservatives. Caprylhydroxamic Acid has the best length (C8) of C chain to promote degradation of cell membrane structures. Caprylhydroxamic Acid may maintain a unionized state in acid to neutral and may perform at a neutral pH. Caprylhydroxamic Acid may have excellent efficacy of fungistatic agents.

In some aspects, the homeopathic composition may comprise Centaurea Cyanus Flower Extract or cornflower extract. Centaurea Cyanus Flower Extract may comprise a skin conditioning agent and astringent that is rich in polyphenols, specifically flavonoids and anthocyanins. Cornflower extract may comprise anti-inflammatory properties, which may be effective on or around an injured area.

In some embodiments, the homeopathic composition may comprise Chelidonium Majus or swallow wort, which may commonly be used for treating warts. Swallow wort has appreciable tissue rejuvenating properties. The juice of Chelidonium majus has anti-bacterial/anti-fungal effect, which may be effective to slow or stop excessive bleeding.

In some aspects, the homeopathic composition may comprise Citrus Aurantium Amara (Bitter Orange) Flower Water may comprise flavonoids, which are anti-inflammatory, antibacterial, and antifungal. Orange flower water contains hydrophilic properties and may act as a natural surfactant and emulsifier to enhance the application of skin lotions and other cosmetics. In some aspects, the orange flower water may be safe to use directly on the skin, without requiring dilution, and may be used on blotchy, irritated skin. Orange flower water is smoothing, soothing, and brightening due to the skin regenerative and vitamin-rich properties of organic citrus flowers.

In some embodiments, the homeopathic composition may comprise Lauric Acid. Lauric Acid is a main fatty acid in coconut oil, which may be used as a moisturizer for the skin and may reduce redness and flaking of skin with minimal adverse side effects.

In some aspects, the homeopathic composition may comprise Citrus aurantium dulcis (orange) peel oil, which may be extracted from the peels of both sweet and sour oranges by cold compression. Orange oil extract may have inflammatory qualities, which may detoxify congested skin and soothe dry or irritated skin.

In some embodiments, the homeopathic composition may comprise Coenzyme A, which may be adapted from pantothenic acid and adenosine triphosphate. Biologically speaking, this coenzyme plays a vital role in the synthesis and oxidation of fatty acids. Coenzyme A may function as a skin conditioner, emollient, and solvent, and in some aspects, coenzyme A may stimulate collagen production.

In some aspects, the homeopathic composition may comprise Dehydroacetic Acid. As a mild acid, dehydroacetic acid may work with benzyl alcohol as a preservative and antimicrobial to provide a broad protection from contamination. Dehydroacetic acid may function as a fungicide and/or bactericide, which, in some embodiments, may be used in personal care products at a maximum concentration of 0.6%.

In some embodiments, the homeopathic composition may comprise Ginkgo Biloba Leaf Extract, which may be a potent antioxidant that may improve blood flow. Improved blood flow may allow for more effective pain reduction and healing around a site of SRI.

In some aspects, the homeopathic composition may comprise Gluconolactone, which is an ester of gluconic acid and is composed of multiple water-attracting hydroxyl groups. Gluconolactone may hydrate skin and enhance the degree of moisturization of the homeopathic composition. Gluconolactone is a polyhydroxy acid (PHA) that is capable of chelating metals and may also function by scavenging free radicals. In some embodiments, gluconolactone may be directly applied on sensitive skin.

In some embodiments, the homeopathic composition may comprise Hydrolyzed Lupine Protein, which is a hydrolyzed protein from the seeds of the lupine plant (lupinus). The seeds are high in proteins and contain 35-45% of all the essential amino acids. The hydrolyzed lupine protein may have a restructuring and regenerating effect on skin. Lupine peptides are a blend of penta- and hexa-peptides derived from lupin flower protein that may function as an MMP (matrix metalloprotease) inhibitor, which may prevent MMP activity following exposure of the skin to sunlight. Accordingly, the lupine peptides blend may prevent collagen and elastin (the main proteins making up connective tissue) breakdown catalyzed by MMP activity, which may facilitate renewal of the skin's outer layer, which may stimulate the production of collagen & elastin.

In some aspects, the homeopathic composition may comprise Hydroxypropyl tetrahydropyrantriol, which is a sugar-protein hybrid made from xylose, a sugar found abundantly in beech trees. As a series of amino acids, Hydroxypropyl tetrahydropyrantriol is small enough to penetrate the skin, which may allow for deeper and more effective penetration of the homeopathic composition. Hydroxypropyl tetrahydropyrantriol stimulates the production of glycosaminoglycan's (GAGs), or mucopolysaccharides. GAGs, which are an important component of connective tissue and may increase production on an extracellular matrix.

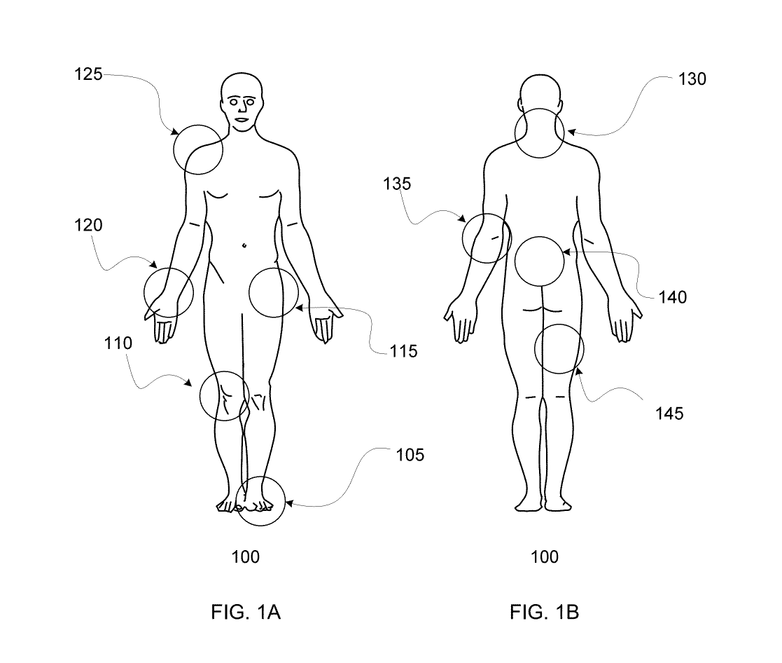

Referring now to FIG. 1A, an anterior view of a human body 100 is illustrated, wherein common sport related injury sites are highlighted. Common sports injuries may comprise a bone or joint injury, such as a shoulder 125 dislocation, twisted ankle 105, knee 110 injury, broken wrist 120, or a displaced hip 115.

Referring now to FIG. 1B, a posterior view of a human body 100 is illustrated, wherein common sport related injury sites are highlighted. Sports injuries may comprise muscle or nerve damage, such as a torn hamstring 145, tennis elbow 135, lower back 140 strain, or neck 130 injury.

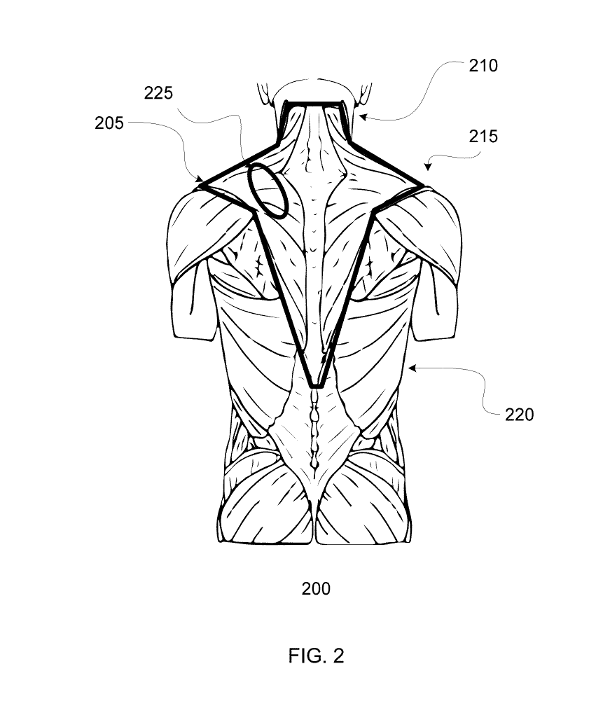

Referring now to FIG. 2, exemplary musculature of the posterior of the human torso 200 is illustrated, wherein the trapezius muscle 205 is highlighted. In some cases, a person may strain or tear his trapezius muscle 205 at a specific damage site 225, such as by dislocating his shoulder 215, poor posture, or stress. The pain may resonate from the neck 210 to shoulder 215 to lower back 220. In some embodiments the homeopathic composition may be applied directly at the damage site 225. In some aspects, the homeopathic composition may be applied over the trapezius muscle 205. In still further implementations, the homeopathic composition may be applied to pain sites, such as the neck 210, shoulder 215, or back 220.

In some aspects, the topical homeopathic composition may be applied to an extended area surrounding the point of the SRI. For example, where the SRI comprises a torn calf muscle, the topical homeopathic composition may be applied from the ankle to the thigh.

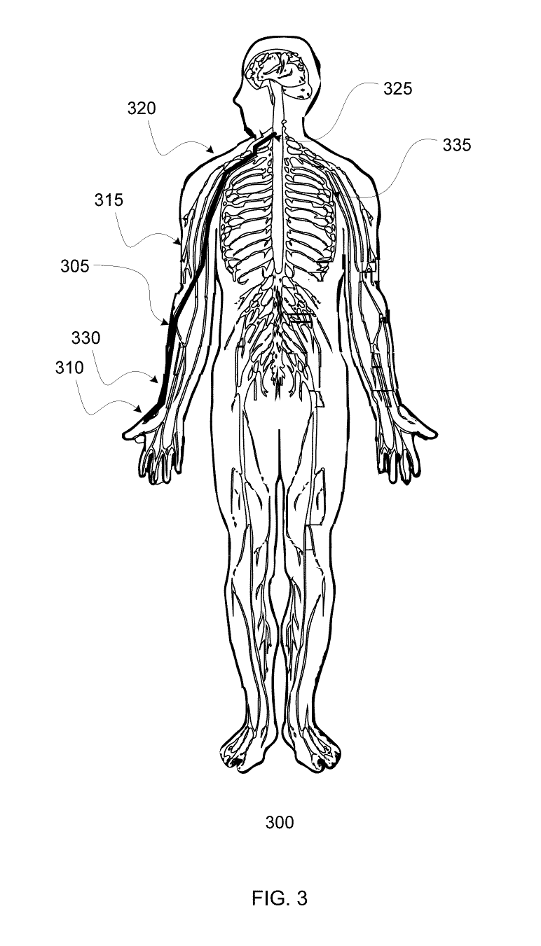

Referring now to FIG. 3, an exemplary nervous system of the human body 300 is illustrated, wherein the radial nerve 305 is highlighted. In some cases, a sports injury may comprise a pinched nerve, such as carpal tunnel syndrome, which may occur from a fracture in the wrist 330, for example. In some aspects, the topical homeopathic composition may be applied along nerve lines associated with the SRI.

For example, the radial nerve 305 extends from the thumb 310 to the upper back 335 in the central nervous system. In some aspects, damage to the radial nerve may cause tennis elbow. In such examples, the topical homeopathic composition may be applied throughout the extension of the radial nerve 305 from thumb 310 to upper back 335, which may include the arm 315 and shoulder 320.

In some aspects, the topical homeopathic composition may be applied at a region of the central nervous system that may be associated with the peripheral nerves that may be affected by the SRI. For example, the radial nerve 305 extends from the central nervous system at the neck 325. In such example, the homeopathic composition may be applied on the neck 325.

In some aspects, the topical treatment may be paired with a second therapy, such as cortisone shots, massages, or surgery. In some such examples, the topical treatment may be a preparation step, wherein a patient may be advised to apply the treatment within a specified period prior to the secondary treatment. The preparation step may reduce the pain caused by the SRI and the pain that may be caused during administration of the treatment.

In other aspects, the topical treatment may be applied during a secondary treatment. Some therapies, such as a massage or ultrasound therapy, may require pressure applied to the area of the SRI, and the topical treatment may help alleviate the pain that may be caused by the pressure. In still further aspects, the topical treatment may be applied after a secondary treatment, wherein the application of the topical homeopathic composition may extend pain relief from the secondary therapy or address pain that may be exacerbated during treatment, such as after physiotherapy.

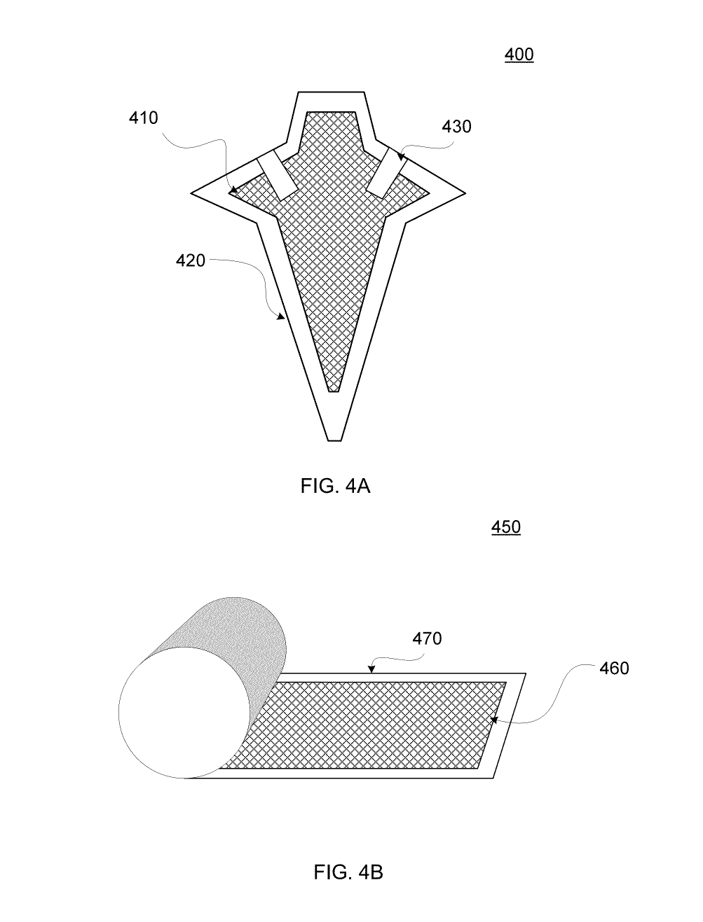

Referring now to FIG. 4A, an exemplary SRI pad 400 with a homeopathic composition 410 is illustrated. In some embodiments, such as illustrated and described in FIG. 2, an SRI may occur on a muscle on the back, such as the trapezius. In some aspects, an SRI pad 400 may be pretreated with a homeopathic composition 460, wherein placing the SRI pad 400 on an SRI site may allow contact and absorption of the homeopathic composition 460 with the skin. In some embodiments, an adhesive portion 420 may allow the SRI pad 400 to stick to the skin without requiring constant pressure. In some aspects, the SRI pad 400 may further comprise securing tabs 430, which may allow for a tighter fit of the SRI pad 400 to the skin. The securing tabs 430 may cause a pain relieving pressure.

Referring now to FIG. 4B, an exemplary roll of SRI wrap 450 with a homeopathic composition 460 is illustrated. In some embodiments, the SRI wrap 450 may comprise a homeopathic composition 460, which may be absorbed when the SRI wrap 450 may be placed against the SRI site. An SRI wrap 450 may be preferable over an SRI pad 400, such as illustrated in FIG. 4A, where the SRI may occur in an area that may be conducive for compression, such as a muscle tear on the leg or arm or a pinched nerve along the arm, such as illustrated and described in FIG. 3. In some embodiments, the borders of the SRI wrap 450 may comprise an adhesive portion 470, which may secure the SRI wrap 450 to the skin and allow for effective absorption of the homeopathic composition 460.

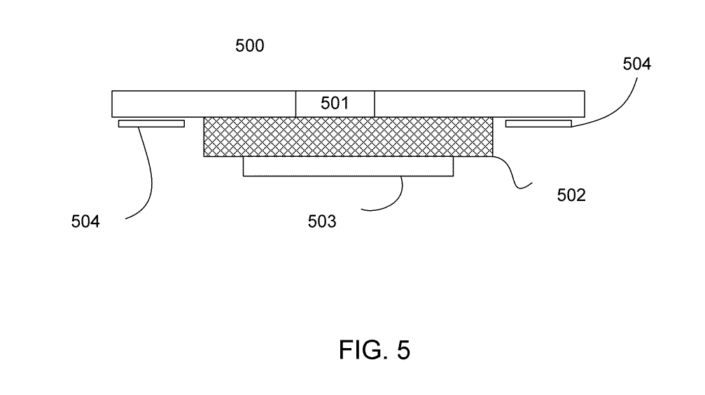

Referring now to FIG. 5, in some embodiments, a treatment bandage 500 may include a substrate 502 dosed with the homeopathic composition 503. The substrate 502 may be comprised of a porous portion impregnated with homeopathic composition 503. The substrate 502 may therefore include one or both of a natural fiber, such as a cotton or other plant based fiber and a synthetic fiber or gel. The substrate 502 may also include a dissolvable starch that may dissolves into the skin. The substrate 502 may be fixedly attached to a sealing layer 501 that generally provides a boundary between the homeopathic composition 503 and an ambient environment. The sealing layer 501 may include a polymer, vinyl, latex, plastic or other material formable to a contour of a human body part.

In some embodiments, the sealing layer may include an adhesive layer 504 for removeably attaching the substrate to a body part in a fashion that allows the homeopathic composition 503 to be brought into contact with the skin of a patient and maintained in contact with the skin. The sealing layer allows that homeopathic composition 503 to remain relatively undisturbed by an ambient environment.

### Illustrative Examples

Illustrative examples of homeopathic composition formulas are listed and described below. These are exemplary only and should not be considered as limiting.

### Example 1

A homeopathic gel prepared by admixing the following ingredients.

| Ingredient | Wt. % |
|---|---|
| Carbomer 940 | 2.10 |
| Xantham gum | 0.15 |
| Propylene glycol | 51.94 |
| Dipropylene glycol | 10.00 |
| Ethoxydiglycol | 15.00 |
| Dimethylisosorbide | 10.00 |
| Aloe Vera gel | 2.00 |
| Surfactant | 0.05 |
| Amica Montana | 2.50 |
| Belladooa | 2.50 |
| Rhus toxicodendron | 2.00 |
| Aescuius hippocastanum | 1.76 |

Although the specific activity of each of either plants or herbs have been recognized, it has been surprisingly found that the combination as now claimed has been found to produce the desired effect. The composition is applied to the limb having pain 1-8 times per day.

### Example 2

A creme was formed by admixing the following ingredients:

| Ingredient | | |
|---|---|---|
| Aesculus hippocastanum extract | 6 | mcg/g |
| Amica Montana extract | 6 | mcg/g |
| L-arginine | 6 | mcg/g |
| Echinacea augustfolia extract | 6 | mcg/g |
| Rhus toxicondendron extract | 6 | mcglg |
| Ruta graveoleus extract | 6 | mcg/g |
| Graphites | 6 | mcg/g |
| Crotalus horridus extract | 0.08 | mcg/g |
| Heloderma horridum extract | 0.08 | mcg/g |
| Lachesis extract | 0.08 | mcg/g |
| Naja extract | 0.08 | mcg/g |
| Isopropyl myristate | 30 | mg/g |
| Brij 72 | 2 | mg/g |
| Brij 72IS | 2 | mg/g |
| Water | 83 | mg/g |
| Methyl paraben | 0.2 | mg/g |
| Propyl paraben | 20 | mcg/g |
| Glycerine | 3 | mg/g |
| 20% NaOH | 3 | mg/g |
| Dowcil 200 | 50 | mcg/g |
| Akodel 112 | 2 | mg/g |

The creme may be used to treat cracked skin on feet or anhidrosis, which is associated with CRPS.

### Example 3

A lotion is prepared by admixing the following ingredients:

| Ingredient | Wt. % |
|---|---|
| Ginger | 1.10 |
| Propylene Glycol Stearate | 6.50 |
| Isocetyl alcohol | 5.00 |
| PEG-100 Stearate | 1.20 |
| Water | 69.90 |
| Echinacea augustfolia extract | 3.00 |
| Methyl paraben | 0.20 |
| Propylene glycol | 12.00 |
| Sorbitan palmitate | 0.60 |
| Arnica Montana | 3.00 |
| Aesculus hippocastanum extract | 2.00 |
| Barberry | 1.00 |
| Mate extract | 0.50 |

If desired, 3% by weight capsaicin can be added. The lotion can be used to treat a patient suffering from fibromyalgia.

### Example 4

### Preparation of a Gel

| Ingredient | Wt. % |
|---|---|
| Arnica Montana | 5.00 |
| Primrose oil | 3.00 |
| Arginine base (10% solution) | 5.00 |
| (Ajinomoto) | |
| Carbopol 940 | 0.40 |
| Butylene glycol | 6.50 |
| Echinacea augustfolia | 3.00 |
| Chamomile glycolic extract | 3.00 |
| Crotalus horridus venom | 0.50 |
| Preservative | 0.10 |
| Fragrance | 0.10 |
| Deionized water | g.s. |

To 20 ml of water with stirring is added the Carbopol 940. The mixture is stirred until hydration is complete and then butylene glycol is added. The arginine base is then added to the mixture. The remaining ingredients are mixed together and added to the first mixture. The mixing is continued until uniform. The composition can be applied 1-8 times daily to reduce the pain resulting from CRPS.

### Example 5

A gel is prepared by admixing the following ingredients:

| Ingredient | Wt. % |
|---|---|
| Propylene Glycol | 43 |
| Polyacrylic acid | 2.1 |
| Dipropylene Glycol | 16 |
| Xantbam Gum | 0.15 |
| Etboxydiglycol | 15 |
| Dimetbylisosorbide | 10 |
| Ascorbic Acid | 2 |
| Chloroxylenol | 0.2 |
| Linoleamidopropyl PG- | 1.5 |
| diammonium chloride phosphate | |
| Glyceretb 4.5 Lactate | 2 |
| Naja venom extract | 2 |
| Echinacea augustfolia | 2 |
| Octoxynol-9 | 0.5 |
| Primrose Oil | 2 |
| Cocamidopropyl PG-dimon | 1 |
| chloride phosphate | |
| Water | 6 |
| Ginger | 0.44 |

Ingredients 1 and 2 are mixed to disperse and form a gel. About 80% of ingredient 3 is mixed with ingredient 4, added to the gel slightly heated with admixture. The balance of 3 is mixed with ingredients 5-17 and added to the gel at 38 degrees. After mixing, the pH is adjusted to about 4 and then the gel is brought to room temperature. Arnica Montana may also be added to have a plurality of dual acting ingredients. Capsaicin together with the ginger can be used to relieve pain.

### CONCLUSION

A number of embodiments of the present invention have been described. While this specification contains many specific implementation details, there should not be construed as limitations on the scope of any inventions or of what may be claimed, but rather as descriptions of features specific to particular embodiments of the present invention.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

While the invention has been described in conjunction with specific embodiments, it is evident that many alternatives, modifications and variations will be apparent to those skilled in the art in light of the foregoing description. Accordingly, this description is intended to embrace all such alternatives, modifications, and variations as fall within its spirit and scope.

Although shown and described in what is believed to be the most practical and preferred embodiments, it may be apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims

What is claimed is:

1. A method for alleviating discomfort associated with a sports related injury, the method comprising the steps of:
identifying a situs of sports related injury on human body; and
applying a composition onto the situs of sports related injury on human body, wherein the composition comprises an effective amount of a combination of herbs or extracts of Arnica Montana, Rhus toxicodendron and Aesculus hippocastanum mixed with an effective amount of belladona, in a pharmaceutically acceptable carrier.

2. The method of claim 1 wherein the composition comprises a gel.

3. The method of claim 1 wherein the composition comprises a lotion.

4. The method of claim 1 wherein the composition comprises a cream.

5. The method of claim 1 additionally comprising the step of applying treatment bandage comprising a layer of the composition over the situs of sports related injury on human body.

6. A treatment bandage for alleviating discomfort associated with sports related injury on human body, the treatment bandage comprising:
a sealing layer formable to a contour of the situs of sports related injury on human body;
a substrate fixedly attached to the sealing layer; and
a treating composition one or both of within and on the surface of the substrate fixedly attached to the sealing layer, the treating composition comprising an effective amount of a combination of herbs or extracts of Arnica Montana, Rhus toxicodendron and Aesculus hippocastanum mixed with an effective amount of belladona, in a pharmaceutically acceptable carrier.

7. The treatment bandage of claim 6 wherein the composition comprises a gel.

8. The treatment bandage of claim 6 wherein the composition comprises a lotion.

9. The treatment bandage of claim 6 wherein the composition comprises a cream.

10. The treatment bandage of claim 6 additionally comprising a sealing layer for removably attaching the substrate and treating composition in a position proximate and in contact the situs.

11. The treatment bandage of claim 6, wherein the treatment bandage comprises a roll.

12. The treatment bandage of claim 6, wherein the treatment bandage comprises a shape and size comparable to the situs of sports related injury.
